(19) United States
(12) Patent Application Publication
(10) Pub. No.: US 2026/0041373 A1
(43) Pub. Date: Feb. 12, 2026

(54) BIOSENSOR

(71) Applicant: ULTRAE CO., LTD., Taichung (TW)

(72) Inventors: Hsieh-Hsun CHUNG, Taichung (TW); Chun-Chieh WANG, Taichung (TW)

(73) Assignee: ULTRAE CO., LTD., Taichung (TW)

(21) Appl. No.: 18/800,113

(22) Filed: Aug. 11, 2024

(51) Int. Cl. A61B 5/00; A61B 5/1473

(52) U.S. Cl. CPC A61B 5/6849; A61B 5/1473; A61B 2562/028

(57) ABSTRACT

Embodiments of the present invention relate to a biosensor. The biosensor includes a main body, a spring, a shell, a biosensing unit, a base, and a push member. The spring is configured to be disposed on the main body. The shell is configured to couple to a first side of the main body. The biosensing unit is configured to couple to a second side of the main body and is disposable. The base is configured to couple to the shell to accommodate the main body and the biosensing unit between the shell and the base. The push member defines a needle receiver for removably coupling to a needle.

### BACKGROUND

Unless otherwise indicated herein, the approaches described in this section are not prior art to the claims in this application and are not admitted to be prior art by inclusion in this section.

A biosensor is configured to insert a test line into the skin of a host being tested. A conventional biosensor includes multiple components and is often made to be disposable. However, only some components of the conventional biosensor are used in a test. The remaining components, which are not used in the test, are still disposed of after each test, resulting in unnecessary waste. In addition, a conventional test line in the conventional biosensor is implanted into the skin of a host when a needle is inserted into the skin. However, since the needle and the conventional test line are both directly in contact with the skin, the conventional test line is often deformed or damaged in the insertion process which may cause undesired contaminations. Therefore, there is a need to provide a new biosensor to overcome at least the shortcomings set forth above.

### BRIEF DESCRIPTION OF THE DRAWINGS

The foregoing and other features of the present disclosure will become more fully apparent from the following description and appended claims, taken in conjunction with the accompanying drawings. These drawings depict only several embodiments in accordance with the disclosure and are therefore not to be considered limiting of its scope. The disclosure will be described with additional specificity and detail through use of the accompanying drawings.

FIG. 1A illustrates a perspective view of biosensor 100 according to some embodiments of the present disclosure.

FIG. 1B illustrates an exploded perspective view of biosensor 100 according to some embodiments of the present disclosure.

FIG. 2A illustrates a bottom perspective view of main body 201 of a biosensor according to some embodiments of the present disclosure.

FIG. 2B illustrates a top perspective view of main body 201 of a biosensor according to some embodiments of the present disclosure.

FIG. 3A illustrates a bottom perspective view of shell 302 of a biosensor according to some embodiments of the present disclosure.

FIG. 3B illustrates a top perspective view of shell 302 of a biosensor, according to some embodiments of the present disclosure.

FIG. 4 illustrates an exploded perspective view of main body 401 and assembly tool 406 according to some embodiments of the present disclosure.

FIG. 5A illustrates a bottom perspective view of assembly tool 506 according to some embodiments of the present disclosure.

FIG. 5B illustrates a top perspective view of assembly tool 506 according to some embodiments of the present disclosure.

FIG. 6A illustrates a bottom perspective view of a combination of main body 601 and assembly tool 606 according to some embodiments of the present disclosure.

FIG. 6B illustrates a top perspective view of a combination of main body 601 and assembly tool 606 according to some embodiments of the present disclosure.

FIG. 7A illustrates an exploded perspective view of shell 702 and a combination of main body 701 and assembly tool 706 according to some embodiments of the present disclosure.

FIG. 7B illustrates a perspective view of a combination of main body 701, shell 702 and assembly tool 706 according to some embodiments of the present disclosure.

FIG. 7C illustrates an exploded perspective view of assembly tool 706 and a combination of main body 701 and shell 702 according to some embodiments of the present disclosure.

FIG. 7D illustrates a top perspective view of a combination of main body 701 and shell 702 according to some embodiments of the present disclosure.

FIG. 7E illustrates a bottom perspective view of a combination of main body 701 and shell 702 according to some embodiments of the present disclosure.

FIG. 8A illustrates a perspective view of biosensing unit 803 according to some embodiments of the present disclosure.

FIG. 8B illustrates an exploded perspective view of biosensing unit 803 according to some embodiments of the present disclosure.

FIG. 8C illustrates a perspective view of test line 803-11 according to some embodiments of the present disclosure.

FIG. 8D illustrates a cross-sectional view of one end of test line 803-11 according to some embodiments of the present disclosure.

FIG. 9A illustrates a top perspective view of a combination of main body 901 and biosensing unit 903 according to some embodiments of the present disclosure.

FIG. 9B illustrates a bottom perspective view of a combination of main body 901 and biosensing unit 903 according to some embodiments of the present disclosure.

FIG. 10 illustrates a perspective view of a combination of main body 1001, shell 1002 and biosensing unit 1003 according to some embodiments of the present disclosure.

FIG. 11A illustrates a top perspective view of base 1104 according to some embodiments of the present disclosure.

FIG. 11B illustrates a bottom perspective view of base 1104 according to some embodiments of the present disclosure.

FIG. 12 illustrates an exploded perspective view of base 1204 and a combination of main plate 1201 and shell 1202 according to some embodiments of the present disclosure.

FIG. 13A illustrates a perspective view of push member 1305 according to some embodiments of the present disclosure.

FIG. 13B illustrates an exploded perspective view of push member 1305 according to some embodiments of the present disclosure.

FIG. 14A illustrates an exploded perspective view of push member 1405 and a combination of main body 1401, shell 1402, biosensing unit 1403, and base 1404 according to some embodiments of the present disclosure.

FIG. 14B illustrates a side perspective view of push member 1405 and a combination of main body 1401, shell 1402, biosensing unit 1403, and base 1404 according to some embodiments of the present disclosure.

FIG. 14C illustrates a sectional perspective view of a combination of main body 1401, shell 1402 and push member 1405 according to some embodiments of the present disclosure.

FIG. 15A illustrates a perspective view of needle 1505-4 according to some embodiments of the present disclosure.

FIG. 15B illustrates an enlarged perspective view of needle 1505-4 according to some embodiments of the present disclosure.

FIG. 15C illustrates a side sectional perspective view of needle 1505-4 according to some embodiments of the present disclosure.

FIG. 16 illustrates a sectional perspective view of biosensor 1600 before needle 1605-4 is being inserted into skin according to some embodiments of the present disclosure.

FIG. 17A illustrates a sectional perspective view of biosensor 1700 as needle 1705-4 is being inserted into skin according to some embodiments of the present disclosure.

FIG. 17B illustrates a sectional enlarged perspective view of needle 1705-4 and test line 1703-11 being inserted into skin according to some embodiments of the present disclosure.

FIG. 17C illustrates a sectional perspective view of biosensor 1700 after needle 1705-4 is inserted into a predetermined depth of skin according to some embodiments of the present disclosure.

FIG. 17D illustrates a sectional enlarged perspective view of needle 1705-4 and test line 1703-11 after they are inserted into a predetermined depth of skin according to some embodiments of the present disclosure.

FIG. 18A illustrates a side perspective view of biosensing unit 1803 after being inserted into skin according to some embodiments of the present disclosure.

FIG. 18B illustrates a side sectional perspective view of biosensing unit 1803 after being inserted into skin according to some embodiments of the present disclosure.

### DETAILED DESCRIPTION

In the following detailed description, reference is made to the accompanying drawings, which form a part hereof. In the drawings, similar symbols typically identify similar components and same numerals typically identify same components, unless context dictates otherwise. The illustrative embodiments described in the detailed description and drawings are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated herein.

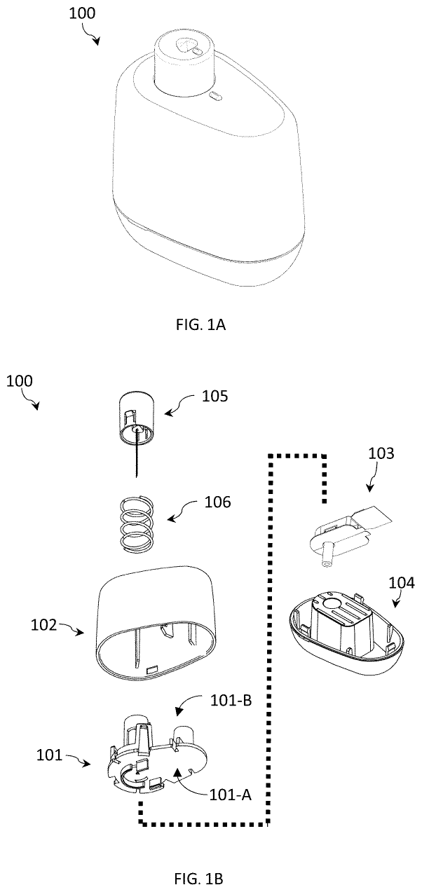

FIG. 1A illustrates a perspective view of biosensor 100 and FIG. 1B illustrates an exploded perspective view of biosensor 100, both arranged in accordance with some embodiments of the present disclosure. In conjunction with FIG. 1A and FIG. 1B, biosensor 100 includes main body 101, shell 102, biosensing unit 103, base 104, push member 105 and spring 106. Shell 102 is configured to couple to one side 101-B of main body 101. Biosensing unit 103 is configured to couple to the other side 101-A of main body 101. Base 104 is configured to couple to shell 102 to accommodate main body 101 and biosensing unit 103 between shell 102 and base 104. Push member 105 is configured to couple to shell 102 and side 101-B of main body 101. Spring 106 is configured to be disposed on main body 101. In some embodiments, main body 101, shell 102, and base 104 are non-disposable and may be used and reused in different tests. In alternative embodiments, biosensing unit 103 is disposable and replaceable after each test. Similarly, a needle (which will be further described in details below) included in push member 105 can also be disposable and replaceable. Push member 105 is configured to press against spring 106 to actuate the needle through main body 101 so that the needle may insert into skin of a host, which may be a human, an animal, or a plant.

In some embodiments, biosensing unit 103 is a multi-electrode electrochemical sensing unit. Biosensing unit 103 is configured to perform invasive detection on a host. Biosensing unit 103 is configured to detect whether the host includes a target analyte, and/or a concentration of the target analyte. The target analyte may be, for example but not limited to, glycated hemoglobin, blood sugar, a heavy metal, a nitrate, a nitrite, an allergen, formaldehyde, dissolved oxygen, uric acid, dopamine, ascorbic acid, potassium ferricyanide, acetaminophen, a halogen ion, a sulfur ion, an aqueous hydrogen peroxide solution, an arsenic(III) ion, a lead ion, a zinc ion, a chromium ion, phenols, an amino acid, or another similar compound.

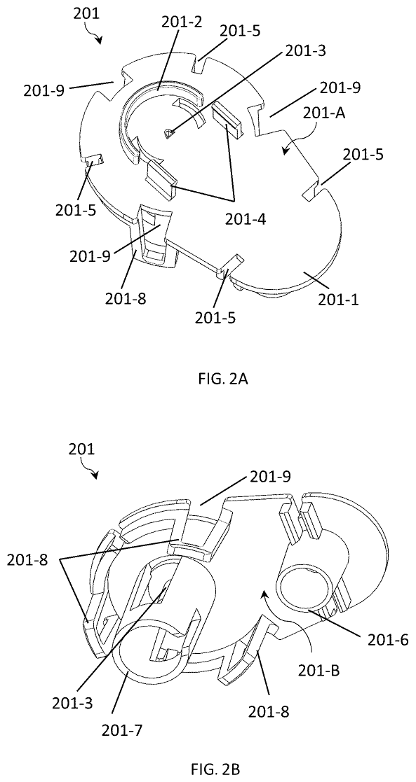

FIG. 2A illustrates a bottom perspective view of main body 201 of a biosensor and FIG. 2B illustrates a top perspective view of main body 201 of biosensor, both arranged in accordance with some embodiments of the present disclosure. In some embodiments, in conjunction with FIG. 1B, main body 201 corresponds to main body 101. In conjunction with FIG. 2A and FIG. 2B, main body 201 includes a main body plate 201-1, a biosensing unit receiver 201-2, a main body tunnel 201-3, a pair of biosensing unit buckles 201-4, at least one fix groove 201-5, a female fix member 201-6, a push member receiver 201-7, at least one male buckle 201-8 and at least one assembly groove 201-9. Biosensing unit receiver 201-2 and biosensing unit buckle 201-4 are formed on one side 201-A (corresponding to side 101-A in FIG. 1B in some embodiments) of main body plate 201-1. Main body tunnel 201-3 is formed on the other side 201-B (corresponding to side 101-B in FIG. 1B in some embodiments) of main body plate 201-1. Main body tunnel 201-3 may be surrounded by push member receiver 201-7.

In some embodiments, at least one fix groove 201-5 is formed at an edge of main body plate 201-1. In some embodiments, there are four fix grooves 201-5 formed at the edge of the main body plate 201-1. Female fix member 201-6 and push member receiver 201-7 are formed on the other side 201-B of main body plate 201-1. The at least one male buckle 201-8 are formed on the other side 201-B and at the edge of the main body plate 201-1. In some embodiments, there are three male buckles 201-8 formed on the other side 201-B and at the edge of main body plate 201-1.

In some embodiments, at least one assembly groove 201-9 is formed at the edge of the main body plate 201-1. In some embodiments, there are three assembly grooves 201-9 formed at the edge of the main body plate 201-1.

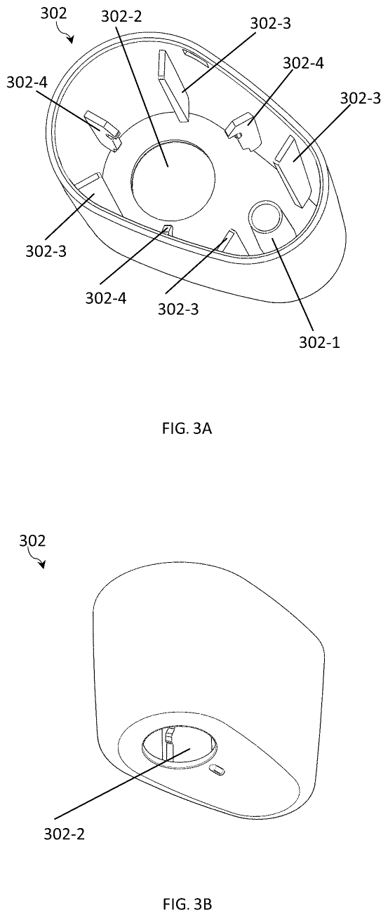

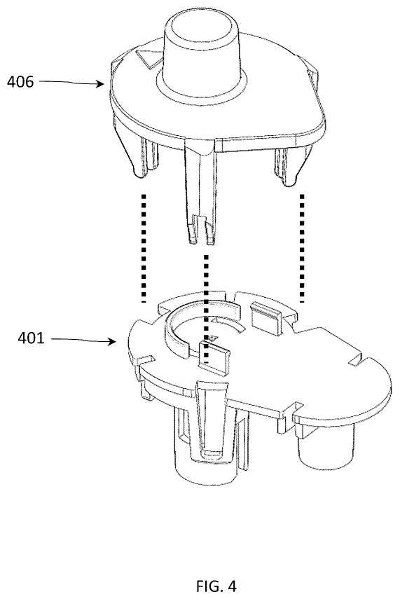

FIG. 3A illustrates a bottom perspective view of shell 302 of a biosensor and FIG. 3B illustrates a top perspective view of shell 302 of biosensor, both arranged in accordance with some embodiments of the present disclosure. In some embodiments, in conjunction with FIG. 1B, shell 302 corresponds to shell 102. In conjunction with FIG. 3A and FIG. 3B, shell 302 may include male fix member 302-1, opening 302-2, at least one positioning element 302-3, and at least one female buckle 302-4. Further, in conjunction with FIG. 2B, male fix member 302-1 is configured to couple with female fix member 201-6. In conjunction with FIG. 2B, opening 302-2 is configured to be passed through by push member receiver 201-7.

In some embodiments, in conjunction with FIG. 2A and FIG. 3A, at least one positioning member 302-3 is configured to couple with the fix groove 201-5 to fix main body 201 in shell 302. In some embodiments, there may be four positioning members 302-3. Any of positioning members 302-3 of is configured to couple with a fix groove 201-5. In some embodiments, in conjunction with FIG. 2B and FIG. 3A, at least one female buckle 302-4 is configured to couple with male buckle 201-8 to fix main body 201 in shell 302.

FIG. 4 illustrates an exploded perspective view of main body 401 and assembly tool 406 according to some embodiments of the present disclosure. In some embodiments, in conjunction with FIG. 1B, main body 401 corresponds to main body 101. In conjunction with FIG. 3A and FIG. 3B, assembly tool 406 is configured to assemble main body 401 into shell 302.

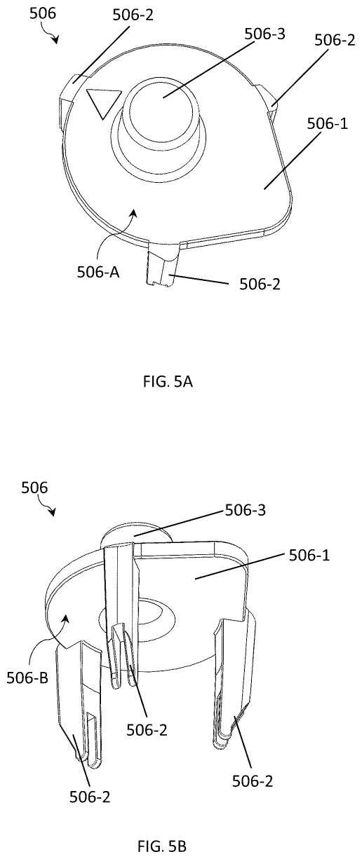

FIG. 5A illustrates a bottom perspective view of assembly tool 506 and FIG. 5B illustrates a top perspective view of assembly tool 506, both arranged in accordance with some embodiments of the present disclosure. In some embodiments, in conjunction with FIG. 4, assembly tool 506 corresponds to assembly tool 406. In conjunction with FIG. 5A and FIG. 5B, assembly tool 506 includes plate 506-1, at least one assembly buckle 506-2, and handle part 506-3. In some embodiments, there are three assembly buckles 506-2 formed on side 506-B. Handle part 506-3 is formed on the other side 506-A of the plate 506-1.

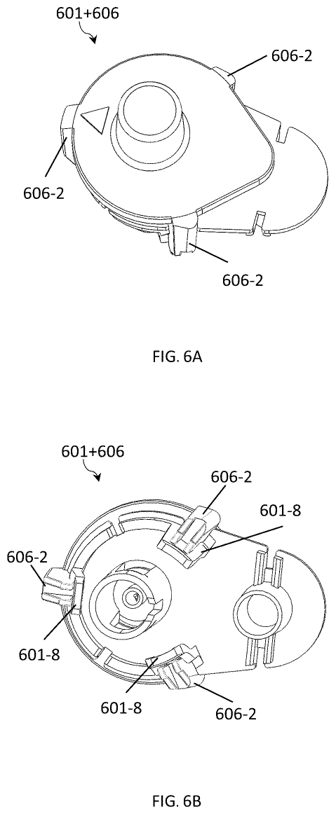

FIG. 6A illustrates a bottom perspective view of a combination of main body 601 and assembly tool 606, and FIG. 6B illustrates a top perspective view of a combination of main body 601 and assembly tool 606, both arranged in accordance with some embodiments of the present disclosure. In some embodiments, in conjunction with FIG. 4, main body 601 corresponds to main body 401, and assembly tool 606 corresponds to assembly tool 406.

In some embodiments, in conjunction with FIG. 2A, FIG. 2B, FIG. 6A and FIG. 6B, at least one assembly buckle 606-2 is configured to pass through at least one assembly groove 201-9 in FIG. 2B. In some embodiments, there are three assembly buckles 606-2, three male buckles 601-8, and three assembly grooves 201-9. Any of three assembly buckles 606-2 is configured to pass through one of assembly grooves 201-9 shown in the FIG. 2B to couple with one of male buckles 601-8 of main body 601.

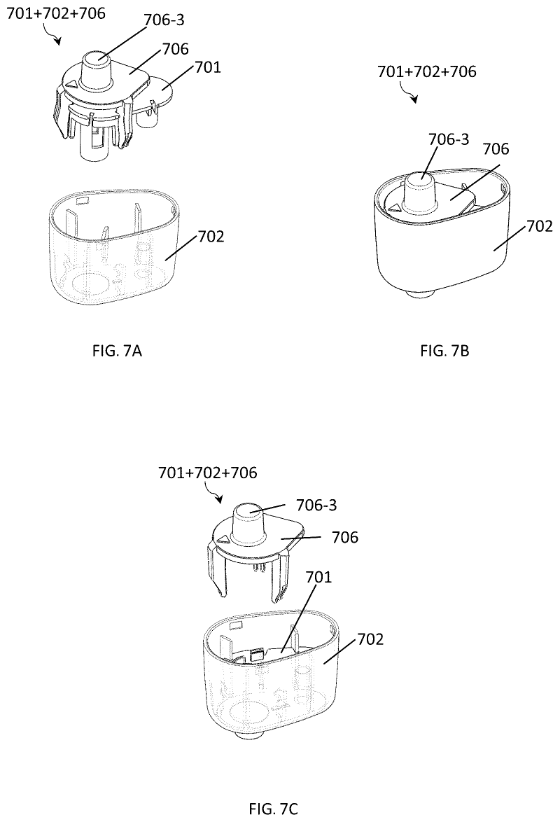

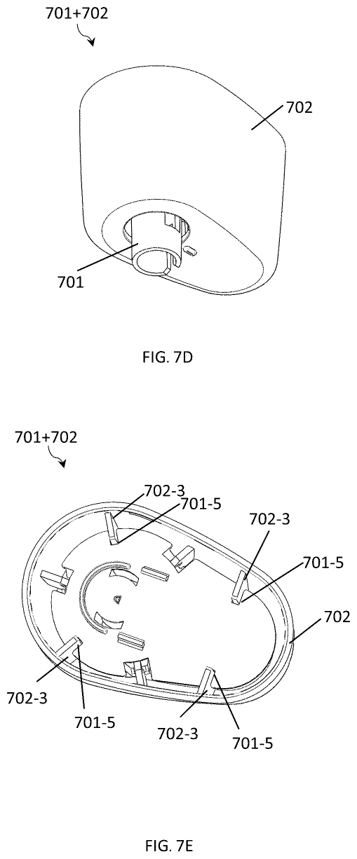

FIG. 7A illustrates an exploded perspective view of shell 702 and a combination of main body 701 and assembly tool 706, FIG. 7B illustrates a perspective view of a combination of main body 701, shell 702 and assembly tool 706, and FIG. 7C illustrates an exploded perspective view of assembly tool 706 and a combination of main body 701 and shell 702, all arranged in accordance with some embodiments of the present disclosure. In some embodiments, in conjunction with FIGS. 6A and 6B, the combination of main body 701 and assembly tool 706 corresponds to the combination of main body 601 and assembly tool 606, and assembly tool 706 corresponds to assembly tool 606.

In some embodiments, in conjunction with FIG. 7A - 7C, assembly tool 706 is configured to assist coupling of main body 701 and shell 702. In some embodiments, first, main body 701 is combined with assembly tool 706 as illustrated in FIG. 7A. Then, in some embodiments, the combination of main body 701 and assembly tool 706 is pushed in shell 702 as illustrated in FIG. 7B. More specifically, in some embodiments, in conjunction with FIG. 2A and FIG. 3A, male buckles 201-8 of main body 201/701 is pushed to couple with female buckles 302-4 of shell 302/702. Finally, after main body 701 is coupled with shell 702, handle part 706-3 of assembly tool 706 is pulled to separate assembly tool 706 from main body 701 as illustrated in FIG. 7C.

FIG. 7D illustrates a top perspective view of a combination of main body 701 and shell 702 and FIG. 7E illustrates a bottom perspective view of a combination of main body 701 and shell 702, both arranged in accordance with some embodiments of the present disclosure. In conjunction with FIG. 7D and FIG. 7E, when main body 701 is coupled to shell 702, positioning members 702-3 of shell 702 are aligned with and coupled to fix grooves 701-5 to stably combine main body 701 with shell 702.

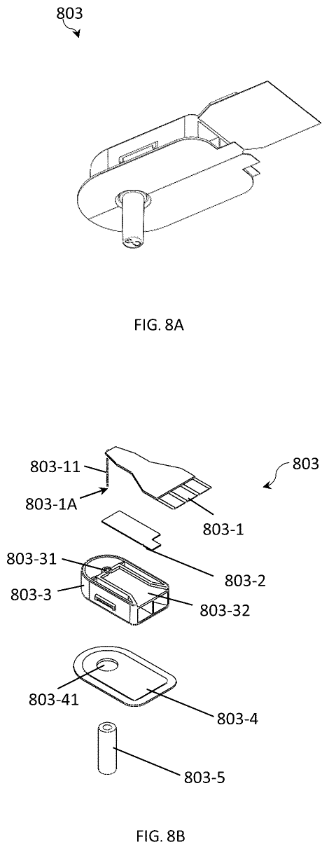

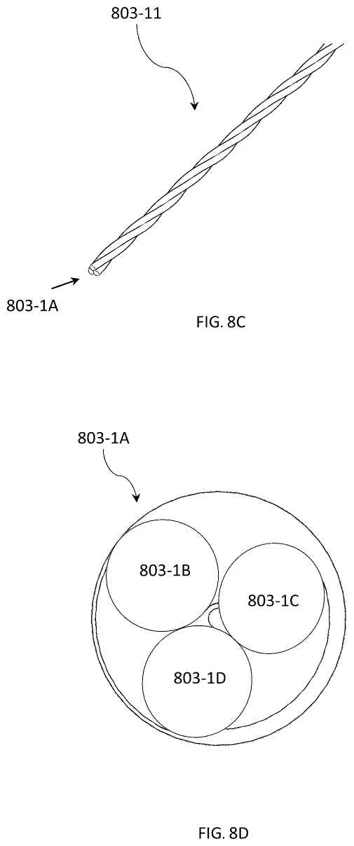

FIG. 8A illustrates a perspective view of biosensing unit 803 and FIG. 8B illustrates an exploded perspective view of biosensing unit 803, both arranged in accordance with some embodiments of the present disclosure. In some embodiments, in conjunction with FIG. 1B, biosensing unit 803 corresponds to biosensing unit 103.

In some embodiments, in conjunction with FIG. 8A and FIG. 8B, biosensing unit 803 includes a microelectrode 803-1, first adhesive 803-2, microelectrode tray 803-3, second adhesive 803-4 and protector 803-5. Microelectrode 803-1 further includes a test line 803-11.

FIG. 8C illustrates a perspective view of test line 803-11 and FIG. 8D illustrates a cross-sectional view of one end of test line 803-11, both arranged in accordance with some embodiments of the present disclosure. In some embodiments, the one end of test line 803-11 is configured to be inserted into skin of a host.

In some embodiments, test line 803-11 is formed by coiling multiple sensor lines. In some embodiment, the diameter of test line 803-11 is less than 0.1 mm. In some embodiments, one end 803-1A of the test line 803-11 comprises free ends 803-1B, 803-1C, and 803-1C of the coiled sensor lines. Free ends 803-1B, 803-1C, and 803-1C may be soft.

In some embodiments, microelectrode tray 803-3 further includes tunnel 803-31 and tray plate 803-32. Test line 803-11 is configured to pass through tunnel 803-31, and the first adhesive 803-2 is attached to tray plate 803-32. Microelectrode 803-1 is disposed on the microelectrode tray 803-3 through first adhesive 803-2. Second adhesive 803-4 defines a hole 803-41. Second adhesive 803-4 is configured to be attached to a bottom side of tray plate 803-32, which is opposite to the side attached with first adhesive 803-2. Hole 803-41 is aligned with tunnel 803-31 for test line 803-11 passing therethrough. Protector 803-5 is configured to couple with hole 803-41 to provide additional protections to test line 803-11 before using.

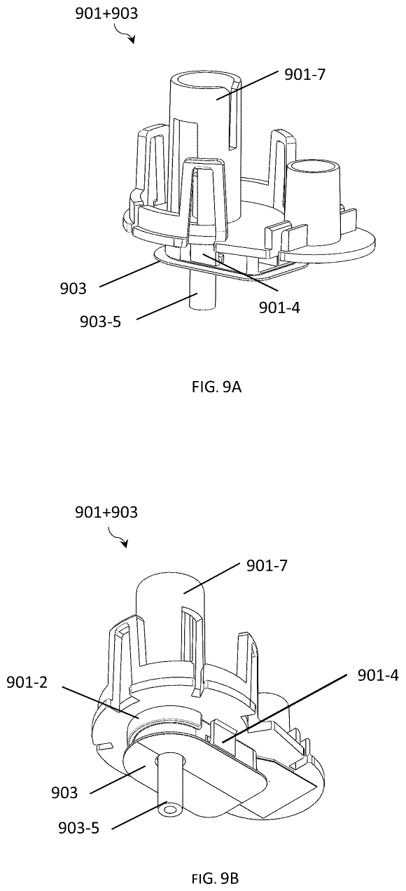

FIG. 9A illustrates a top perspective view of a combination of main body 901 and biosensing unit 903 and FIG. 9B illustrates a bottom perspective view of a combination of main body 901 and biosensing unit 903, both arranged in accordance with some embodiments of the present disclosure. In some embodiments, main body 901 corresponds to main body 101, and biosensing unit 903 corresponds to biosensing unit 103.

In some embodiments, in conjunction with FIG. 2A and FIG. 2B, biosensing unit 903 is positioned in biosensing unit receiver 901-2 (corresponding to the biosensing unit receiver 201-2 in FIG. 2A in some embodiments) and combined with main body 901 by coupling the pair of biosensing unit buckles 901-4 (corresponding to biosensing unit buckles 201-4 shown in FIG. 2A in some embodiments). After biosensing unit 903 combines with main body 901, in conjunction with FIG. 8A and FIG. 8B, the position of tunnel 803-31 in FIG. 8B may correspond to main body tunnel 201-3 in FIG. 2A to allow test line 803-11 passing through main body tunnel 201-3.

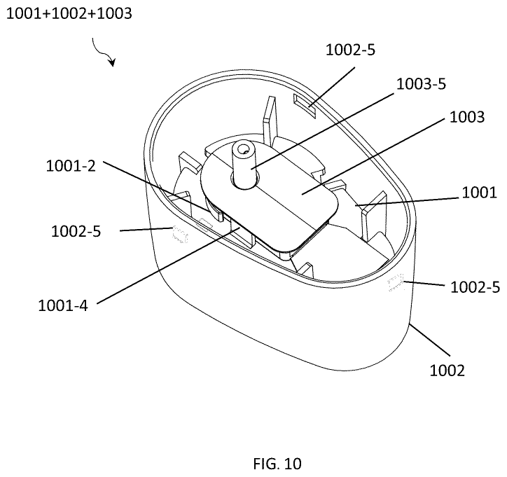

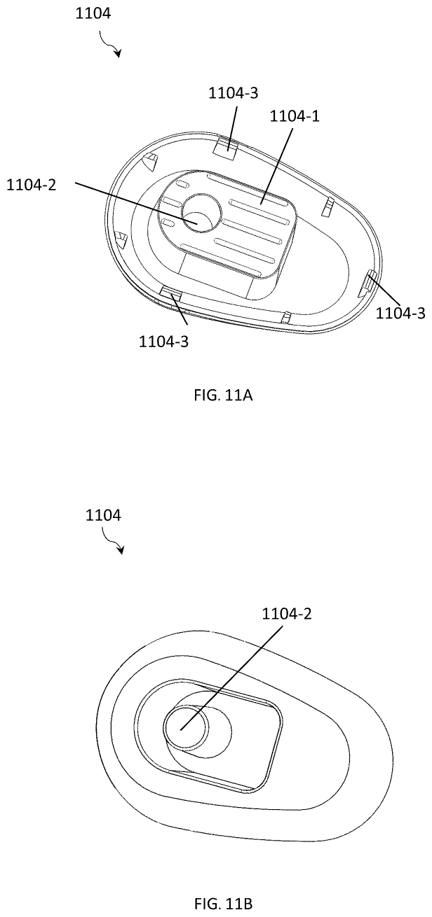

FIG. 10 illustrates a perspective view of a combination of main body 1001, shell 1002, and biosensing unit 1003, arranged in accordance with some embodiments of the present disclosure. In some embodiments, main body 1001 corresponds to main body 101, shell 1002 corresponds to shell 102, and biosensing unit 1003 corresponds to biosensing unit 103. After main body 1001 combines with shell 1002, biosensing unit 1003 is then positioned in biosensing unit receiver 1001-2 and combined with main body 1001 by coupling with biosensing unit buckles 1001-4 (corresponding to biosensing unit buckles 201-4 in some embodiments). In some embodiments, shell 1002 includes one or more shell buckles 1002-5.

FIG. 11A illustrates a top perspective view of base 1104 and FIG. 11B illustrates a bottom perspective view of base 1104, both arranged in accordance with some embodiments of the present disclosure. In conjunction with FIG. 10, base 1104 includes support plate 1104-1, space 1104-2 and one or more base buckles 1104-3. Space 1104-2 is defined in support plate 1104-1 to accommodate protector 1003-5. Base buckles 1104-3 are configured to couple with shell buckles 1002-5.

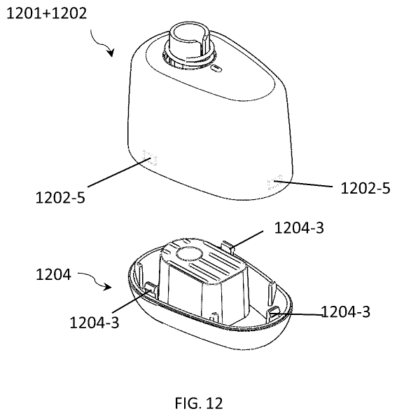

FIG. 12 illustrates an exploded perspective view of base 1204 and a combination of main body 1201 and shell 1202, arranged in accordance with some embodiments of the present disclosure. In some embodiments, in conjunction with FIG. 1B, main body 1201 corresponds to main body 101, shell 1202 corresponds to shell 102, and base 1204 corresponds to base 104.

In some embodiments, base 1204 includes base buckles 1204-3. In conjunction with FIG. 11A, base buckles 1204-3 may correspond to base buckles 1104-3. In some embodiments, base 1204 is configured to couple with the combination of main body 1201 and shell 1202 by coupling base buckles 1204-3 to shell buckles 1202-5.

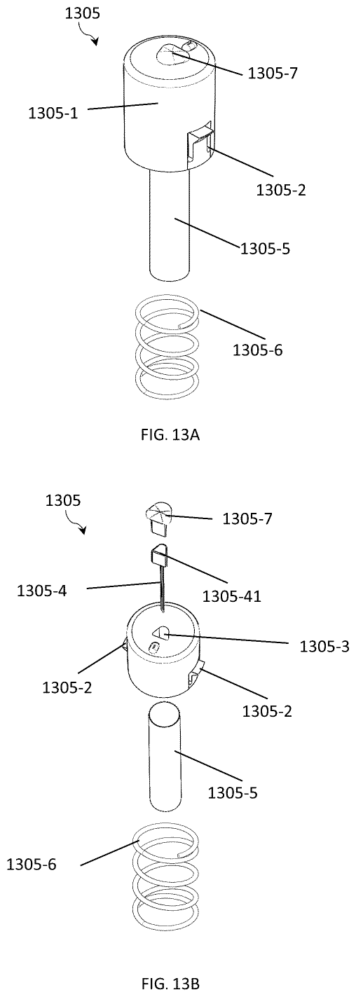

FIG. 13A illustrates a perspective view of push member 1305 and FIG. 13B illustrates an exploded perspective view of push member 1305, both arranged in accordance with some embodiments of the present disclosure. In some embodiments, in conjunction with FIG. 1B, push member 1305 corresponds to push member 105. In some embodiments, push member 1305 includes push element 1305-1, a pair of push element buckles 1305-2, needle 1305-4, needle protector 1305-5, and cap 1305-7. Push member 1305 further defines needle receiver 1305-3 for removably coupling needle 1305-4.

In some embodiments, push element 1305-1 is configured to be pushed by a user. Needle receiver 1305-3 is defined as a tunnel space for removably coupling needle 1305-4. Needle 1305-4 further includes coupling part 1305-41 formed on one end of needle 1305-4. With coupling part 1305-41, in some embodiments, needle 1305-4 can be placed in or removed from needle received 1305-3. Needle protector 1305-5 is configured to prevent needle 1305-4 from being exposed and accidentally injuring a user before push member 1305 is combined with other components of the biosensor.

Cap 1305-7 is configured to couple with coupling part 1305-41 of needle 1305-4 and further secure needle 1305-4, in needle receiver 1305-3, with push element 1305-1. In some embodiments, needle 1305-4 is disposable and replaceable.

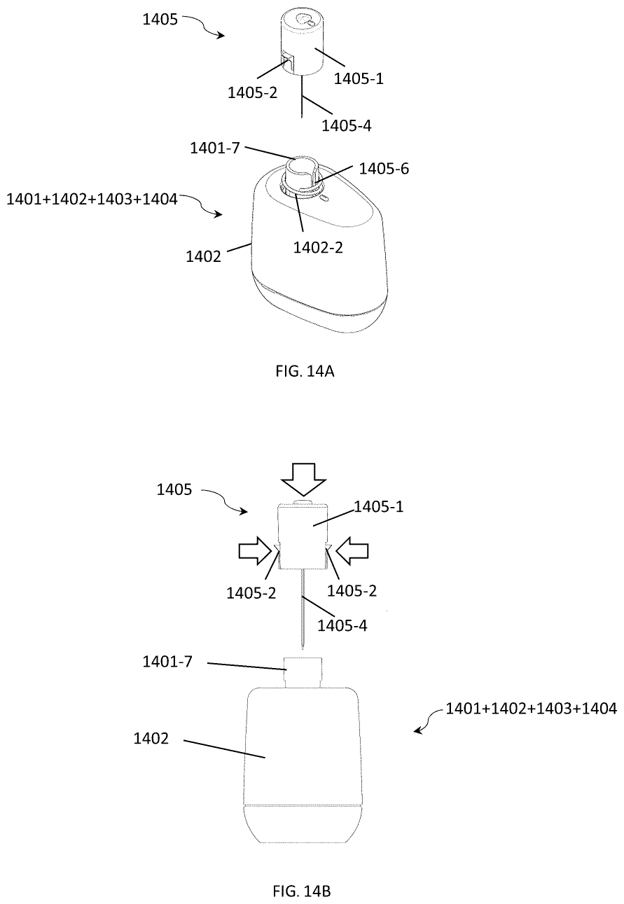

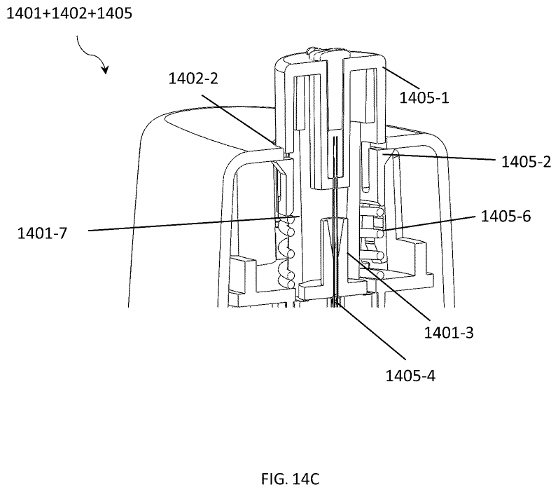

FIG. 14A illustrates an exploded perspective view of push member 1405 and a combination of main body 1401, shell 1402, biosensing unit 1403 and base 1404, and FIG. 14B illustrates a side perspective view of push member 1405 and a combination of main body 1401, shell 1402, biosensing unit 1403 and base 1404, both arranged in accordance with some embodiments of the present disclosure. In some embodiments, in conjunction with FIG. 1B, main body 1401 corresponds to main body 101, shell 1402 corresponds to shell 102, biosensing unit 1403 corresponds to biosensing unit 103, base 1404 corresponds to base 104, and push member 1405 corresponds to push member 105.

FIG. 14C illustrates a sectional perspective view of a combination of main body 1401, shell 1402, and push member 1405 according to some embodiments of the present disclosure. In some embodiments, in conjunction with FIG. 13A and FIG. 13B, spring 1405-6 corresponds to spring 1305-6. In some embodiments, spring 1405-6 is configured to position through opening 1402-2 (corresponding to the opening 302-2 shown in FIG. 3A in some embodiments) and surround push member receiver 1401-7 (corresponding to push member receiver 201-7 shown in FIG. 2B in some embodiments). Then, push member 1405 is configured to press against spring 1405-6 until the pair of push element buckles 1405-2 is coupled with opening 1402-2. In response to push member 1405-4 being pressed against spring 1405-6, needle 1405-4 is configured to be actuated and then pass through push member receiver 1401-7 and main body tunnel 1401-3 (corresponding to the main body tunnel 201-3 shown in FIG. 2A and FIG. 2B in some embodiments). In some embodiments, as shown illustrated in FIG. 14C, a user may further press push element 1405-1 to push against spring 1405-6 to actuate needle 1405-4 through main body tunnel 1401-3.

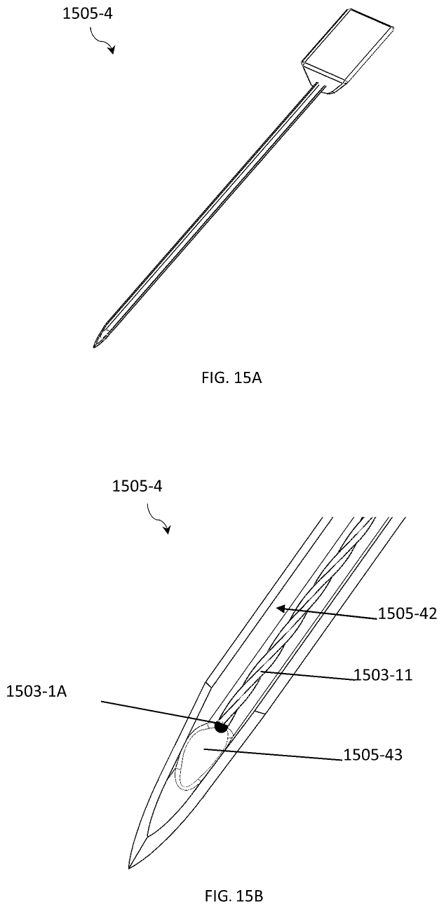

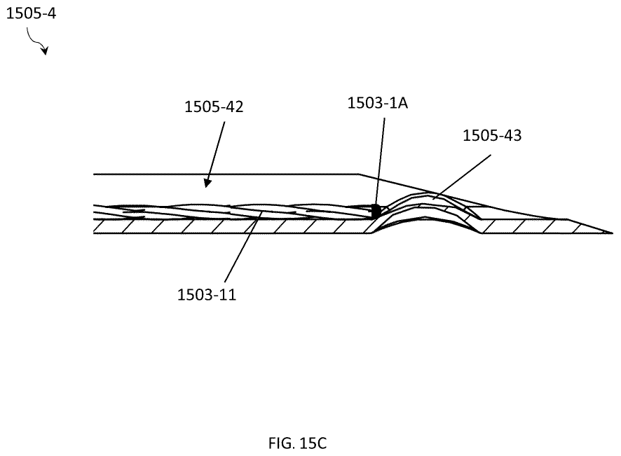

FIG. 15A illustrates a perspective view of needle 1505-4, FIG. 15B illustrates an enlarged perspective view of needle 1505-4, and FIG. 15C illustrates a side sectional view of needle 1505-4, all arranged in accordance with some embodiments of the present disclosure. In some embodiments, in conjunction with FIGS. 13B, 14A, and 14B, needle 1505-4 corresponds to needle 1305-4 or 1405-4.

In some embodiments, needle 1505-4 further defines side groove 1505-42 and further includes protruding area 1505-43. Test line 1503-11 (corresponding to test line 803-11 in FIG. 8B in some embodiments) further includes end 1503-1A. In conjunction with FIGS. 8C and 8D, end 1503-1A may correspond to end 803-1A. End 1503-1A may include free ends of the coiled sensor lines in test line 1503-11. In some embodiments, test line 1503-11 may be received in the side groove 1505-42.

In some embodiments, test line 1503-11 can move smoothly along a surface of protruding area 1505-43 after leaving side groove 1505-42 when end 1503-1A is pressed against protruding area 1505-43. Details of the movements of test line 1503-11 will be further described below.

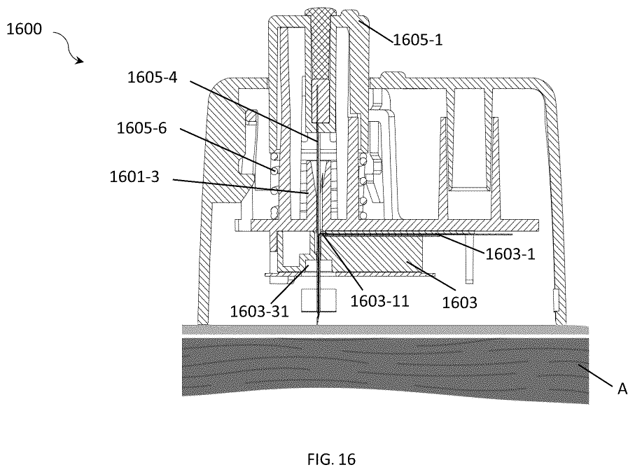

FIG. 16 illustrates a sectional perspective view of biosensor 1600 before needle 1605-4 is being inserted into skin according to some embodiments of the present disclosure. In conjunction with FIG. 12A, before push element 1605-1 (corresponding to push element 1405-1 in FIG. 14A and FIG. 14B in some embodiments) is pressed against spring 1605-6, needle 1605-4 (corresponding to needle 1505-4 in FIG. 15A and FIG. 15B in some embodiments) is configured to pass through main body tunnel 1601-3 (corresponding to main body tunnel 1401-3 in FIG. 14C in some embodiments) and tunnel 1603-31 (corresponding to tunnel 803-31 in FIG. 8B in some embodiments). In addition, test line 1603-11 is also placed in a side groove (not shown) of needle 1605-4. In some embodiments, in conjunction with FIG. 12A, before using biosensor 1600, base 1204 should be removed first. After base 1204 is removed, biosensor 1600 may be placed on skin A for inserting needle 1605-4 and implanting test line 1603-11 into skin A. In some embodiments, needle 1605-4 is configured to be disposed substantially perpendicular to biosensing unit 1603.

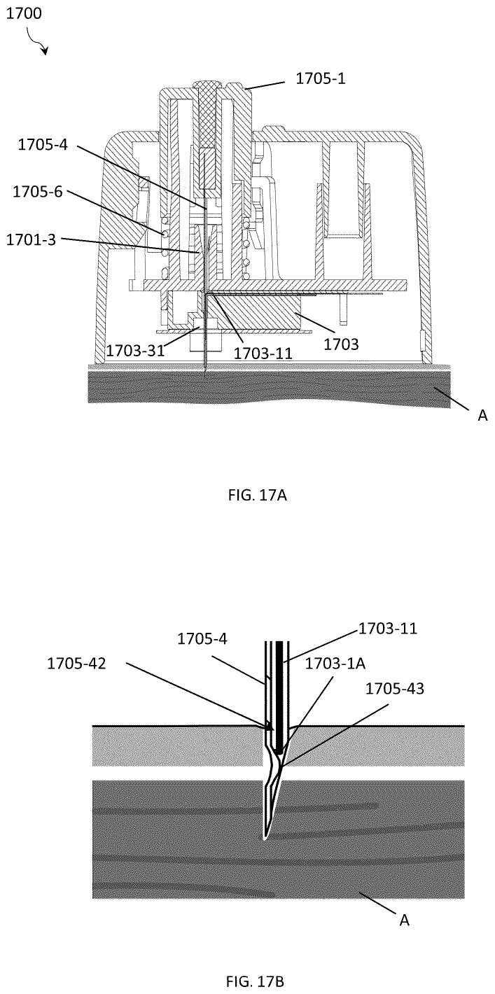

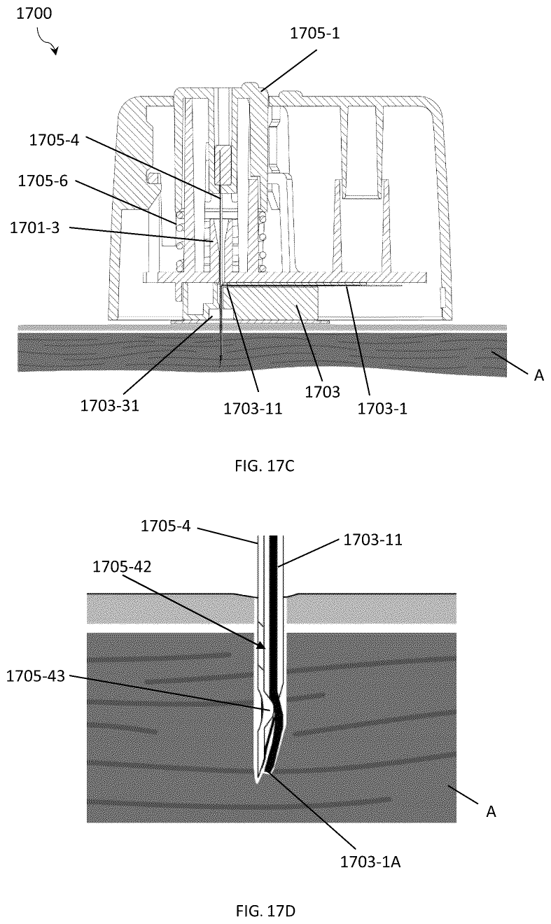

FIG. 17A illustrates a sectional perspective view of biosensor 1700 as needle 1705-4 is being inserted into skin A, and FIG. 17B illustrates a sectional enlarged perspective view of needle 1705-4 and test line 1703-11 being inserted into skin A, both arranged in accordance with some embodiments of the present disclosure. In some embodiments, push element 1705-1 (corresponding to push element 1405-1 in FIG. 14A, FIG. 14B, and FIG. 14C in some embodiments) is pushed to press spring 1705-6 to actuate needle 1705-4 (corresponding to needle 1505-4 in FIG. 15B in some embodiments) forward into skin A and move biosensing unit 1703 to be in direct contact with the surface of skin A. As illustrated in FIG. 17B, needle 1705-4 is inserted into skin A. In response to needle 1705-4 being inserted into skin A, test line 1703-11 is protected by protruding area 1705-43 and side groove 1705-42 from being in direct contact with skin A during the needle insertion. This way, test line 1703-11 can be inserted into skin A smoothly along with needle 1705-4 without being deformed or even damaged during the insertion process.

FIG. 17C illustrates a sectional perspective view of biosensor 1700 after needle 1705-4 is inserted into a predetermined depth of skin A, and FIG. 17D illustrates a sectional enlarged perspective view of needle 1705-4 and test line 1703-11 after they are inserted into a predetermined depth of skin A, both arranged in accordance with some embodiments of the present disclosure.

After needle 1705-4 reaches the predetermined depth of skin A, needle 1705-4 may be pulled out, and pressed spring 1705-6 may return to its unpressed state. After needle 1705-4 is pulled out, biosensing unit 1703 remains attached to skin A by itself. In some embodiments, as shown in FIG. 8B, biosensing unit 1703 is attached to skin A through second adhesive 803-4. During the period of needle 1705-4 being pulled out from skin A, end 1703-1A and test line 1703-11 are configured to move smoothly along a surface of protruding area 1705-43 and exit side groove 1705-42. End 1703-1A may include free ends of the coiled sensor lines in test line 1703-11. After end 1703-1A and test line 1703-11 exit side groove 1705-42, 1703-1A and test line 1703-11 are in direct contact with skin A. In other words, after needle 1705-4 is pulled out of skin A, test line 1703-11 remains implanted in skin A.

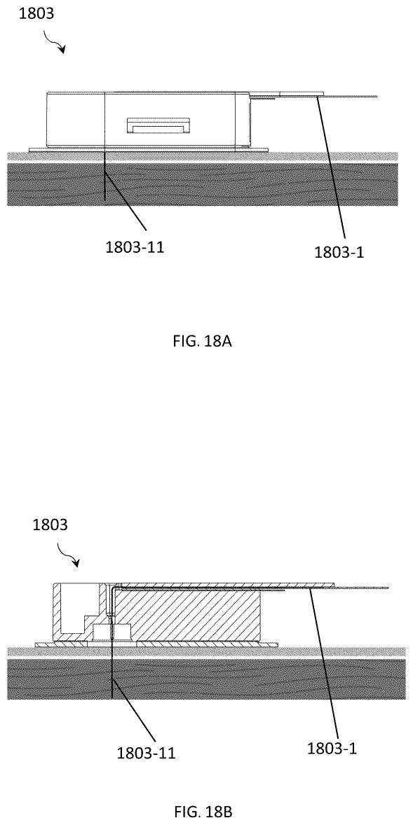

FIG. 18A illustrates a side perspective view of biosensing unit 1803 after being inserted into skin and FIG. 18B illustrates a side sectional perspective view of biosensing unit 1803 after being inserted into skin, both arranged in accordance with some embodiments of the present disclosure. Test line 1803-11 is implanted into skin A to make further measurements and transmit signals to microelectrode 1803-1. In some embodiments, in conjunction with FIG. 8B, microelectrode 1803-1 corresponds to microelectrode 803-1 and test line 1803-11 corresponds to test line 803-1.

In some embodiments, the present disclosure further discloses a method of assembling a biosensor. The biosensor includes a main body, a shell, a base, and a push member. In addition, the biosensor further includes a disposable and replaceable needle, and a disposable and replaceable biosensing unit.

In some embodiments, a needle may be disposed and replaced. In conjunction with FIGS. 17B and 17D, after needle 1705-4 is inserted into and pulled out from skin A in a first test, needle 1705-4 becomes a used needle. In conjunction with FIGS. 13A and 13B, to remove the used needle received in push member 1305, a user may pull cap 1305-7 coupled with coupling part 1305-41 of used needle 1705-4 (corresponding to needle 1305-4 in FIG. 13B in some embodiments) to remove the used needle 1705-4 from push member 1305.

In some embodiments, after removing the used needle 1705-4, the user may couple cap 1305-7 with a new coupling part 1305-41 of a new needle 1305-4, and assemble cap 1305-4, new coupling part 1305-41 and new needle 1305-4 into push element 1305. Accordingly, the biosensor including new needle 1305-4 may be used in another new test. More specifically, except for the used needle and the coupling part, other components in the biosensor may still be used in the new test. In conjunction with FIG. 1, some example other components include, but not limited to, main body 101, shell 102, base 104 and spring 106.

In some embodiments, a biosensing unit may also be disposed and replaced. After a biosensing unit is attached to skin of a host, the biosensing unit becomes a used biosensing unit. In conjunction with FIG. 17C, used biosensing unit 1703 will be left on skin A of the host and therefore be removed from the biosensor.

In some embodiments, after the used biosensing unit 1703 is attached to skin A of the host and in conjunction with FIG. 10, a user may assemble a new biosensing unit 1003 with main body 1001 for another new test. In some embodiments, new biosensing unit 1003 may be placed in biosensing unit receiver 1001-2 and coupled to main body 1001 with biosensing unit buckles 1001-4. In some embodiments, in conjunction with FIGS. 15B and 13B, the assembling of new biosensing unit 1003 may include receiving test line 1503-11 of new biosensing unit 1003 in side groove 1505-42 of new needle 1305-4.

Embodiments of the present invention relate to a biosensor. The biosensor includes a main body, a shell, a biosensing unit, a base, and a push member. The shell is configured to couple to a first side of the main body. The biosensing unit is configured to couple to a second side of the main body and the biosensing unit is disposable. The base is configured to couple to the shell to accommodate the main body and the biosensing unit between the shell and the base. The push member includes a needle. The needle is disposable. Therefore, by disposing a used needle in the push member and a used biosensing unit and replacing with a new needle and a new biosensing unit, embodiments of the present invention provide a biosensing unit which can be used for multiple times, which can prevent unnecessary materials wastes.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Versatile Disk (DVD), a digital tape, a computer memory, etc. ; and a transmission type medium such as a digital and/or an analog communication medium (e.g., a fiber optic cable, a waveguide, a wired communications link, a wireless communication link, etc.).

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

1. A biosensor, comprising:
a main body;
a spring configured to be disposed on the main body;
a shell configured to couple to a first side of the main body;
a biosensing unit configured to couple to a second side of the main body, wherein the biosensing unit is disposable;
a base configured to couple to the shell to accommodate the main body and the biosensing unit between the shell and the base; and
a push member defining a needle receiver for removably coupling to a needle.

2. The biosensor of claim 1, wherein the biosensing unit and the needle are configured to be replaced after a first test, but the main body, the shell, the base, and the spring are configured to continue to be in use in another new test.

3. The biosensor of claim 1, wherein the needle further includes a coupling part at one end of the needle.

4. The biosensor of claim 3, wherein the push member further includes a cap configured to couple with the coupling part and secure the needle in the needle receiver.

5. The biosensor of claim 1, wherein the needle defines a side groove and includes a protruding area.

6. The biosensor of claim 5, wherein the biosensing unit includes a microelectrode having a test line with an end, and the test line includes coiled sensor lines.

7. The biosensor of claim 6, wherein the side groove is configured to receive the test line.

8. The biosensor of claim 7, in response to the needle being inserted into skin of a host, the test line is protected from being in direct contact with the skin by the side groove and the protruding area.

9. The biosensor of claim 7, in response to the needle being pulled out from skin of a host, the test line is configured to move along a surface of the protruding area, exit the side groove, and remain implanted in the skin.

10. The biosensor of claim 1, wherein the push member further includes a needle protector to prevent the needle from being exposed before the push member is combined with other components of the biosensor.

11. The biosensor of claim 1, wherein the needle is configured to pass through a push member receiver and a main body tunnel of the main body.

12. The biosensor of claim 1, wherein the push member further includes a push element configured to press against the spring to actuate the needle passing through the main body.

13. The biosensor of claim 1, wherein the needle is configured to be disposed substantially perpendicular to the biosensing unit.

14. The biosensor of claim 1, wherein the base is configured to be removed from the biosensor before the needle is inserted into skin of a host.

15. A method of using a biosensor having a main body, a shell, a base, and a push member, comprising:
after using the biosensor on a host in a first test, removing a needle in the push member;
including a new needle in the push member; and
using the biosensor including the new needle in another new test.

16. The method of claim 15, wherein the removing further comprises pulling a cap coupled with a coupling part of the needle.

17. The method of claim 16, wherein the including further comprises coupling the cap with a new coupling part of the new needle.

18. The method of claim 15, further comprising:
attaching a biosensing unit from the biosensor to the host in the first test; and
including another new biosensing unit into the biosensor.

19. The method of claim 18, wherein the including another new biosensing unit further comprises placing the another new biosensing unit in a receiver and coupling the new biosensing unit to the main body of the biosensor with buckles.

20. The method of claim 18, wherein the including another new biosensing unit further comprises receiving a test line included in the new biosensing unit in a side groove of the new needle.
